(19) United States
(12) Patent Application Publication
(10) Pub. No.: US 2009/0011507 A1
(43) Pub. Date: Jan. 8, 2009

(54) Scaffolds for Use in Tissue Engineering to Culture Cells

(76) Inventor: Julian Ellis, Southwell (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/168,479

(22) Filed: Jul. 7, 2008

(30) Foreign Application Priority Data
Jul. 7, 2007 (GB) 0713223.6

(51) Int. Cl. C12N 5/06; D05B 3/00

(52) U.S. Cl. 435/395; 112/475.17

(57) ABSTRACT

In the field of tissue engineering there is a need for a scaffold which is suited to low volume manufacture, and which allows the culture of complex cell structures. A scaffold (10, 20, 30), for use in tissue engineering to culture cells, comprises a plurality of interconnected embroidery stitches (12, 14, 32), each stitch (12, 14, 32) being formed from a biocompatible thread (16). A scaffold assembly, for use in tissue engineering to culture cells, comprises a plurality of layers overlying one another, each layer including a scaffold (10, 20, 30) as set out above.

A method of creating a scaffold, for use in tissue engineering to culture cells, comprises the steps of providing a base substrate; and embroidering a plurality of interconnected stitches (12, 14, 32) in the base substrate, each stitch (12, 14, 32) being embroidered using a biocompatible thread (16). A method of creating a scaffold assembly, for use in tissue engineering to culture cells, comprises the step of providing a plurality of layers overlying one another, each layer including a scaffold (10, 20, 30) created as set out above.

This application claims priority under 35 U.S.C. §119(a) to Great Britain Patent Application No. 0713223.6, filed Jul. 7, 2007. The foregoing application is incorporated by reference herein.

### FIELD OF THE INVENTION

This invention relates to a scaffold and a scaffold assembly for use in tissue engineering to culture cells, and a method of making such a scaffold and such a scaffold assembly.

### BACKGROUND OF THE INVENTION

Conventional scaffolds for culturing cells have a textile structure that is woven, non-woven, braided or knitted. These textile structures define a fibrous array which has a shape and form corresponding to the cell structure required.

The shape and form of scaffold required often varies from one application to another and so a given scaffold is normally only required to be produced in a relatively small batch.

However the production of a scaffold having a textile structure involves setting up a corresponding textile machine. This process requires a large quantity of fibre and is time-consuming. As a result it is uneconomic to produce small batches of such scaffolds.

In addition, the aforementioned textile structures provide only a limited variation in spacing between adjacent fibres within the structure. This limits the complexity of cell structures that it is possible to culture on such scaffolds.

Therefore there is a need for a scaffold which is suited to low volume manufacture, and which allows the culture of complex cell structures.

### SUMMARY OF THE INVENTION

According to a first aspect of the invention there is provided a scaffold, for use in tissue engineering to culture cells, comprising a plurality of interconnected embroidery stitches, each stitch being formed from a biocompatible thread.

Embroidery stitches are an assembly of stitches created on a substrate using a needle and thread.

It is possible to create such embroidery stitches without complex and lengthy set up of a machine, and so small batches of scaffolds can be manufactured economically.

In addition, the inclusion of embroidery stitches allows for a wide variation in the spacing of adjacent threads within the scaffold which allows for the culture of complex cell structures.

Preferably the embroidery stitches include one or more of the following: a lock stitch, a chain stitch, a blind stitch, a moss stitch, and a tuft. Such stitches provide the desired variety in spacing of adjacent threads while being readily producible.

The scaffold may include a plurality of tufts having legs of differing lengths. This allows for optimization of the scaffold according to the specific type of cell structure it is desired to culture.

In a preferred embodiment of the invention the biocompatible thread is resorbable. The inclusion of a resorbable biocompatible thread enables the scaffold to be degraded by biological and biochemical processes or dissolve following, for example, implantation in a human or animal body.

In another preferred embodiment of the invention the biocompatible thread is non-resorbable. The inclusion of a non-resorbable biocompatible thread allows the scaffold to remain in a human or animal body following implantation to provide, for example, reinforcement to new cell tissue.

Optionally the biocompatible thread includes a bioactive component to promote the development of a specific type of cell. Providing a bioactive component within a thread allows for the promotion of certain cell types, such as nerve cells or muscle cells, within one or more desired regions of the scaffold.

In another preferred embodiment of the invention the scaffold further includes a base substrate supporting the embroidery stitches. The inclusion of a base substrate helps the scaffold maintain its shape and form during, for example, cell culturing or implantation in a body.

The base substrate may interconnect the embroidery stitches. Such an arrangement allows for an even greater variety in the spacing between adjacent threads within the scaffold by allowing, for example, the inclusion of discrete embroidery stitches or discrete groups of interconnected embroidery stitches within the scaffold.

Optionally the scaffold includes a biocompatible base substrate. The inclusion of a biocompatible base substrate allows the base substrate to remain within a body together with the scaffold following implantation.

Preferably the base substrate is resorbable. Such a base substrate can be degraded by biological and biochemical processes or dissolve following, for example, implantation in a body.

According to a second aspect of the invention there is provided a scaffold assembly, for use in tissue engineering to culture cells, comprising a plurality of layers overlying one another, each layer including a scaffold as described hereinabove.

The provision of such a plurality of layers overlying one another allows the scaffold assembly to adopt a three-dimensional form, and thereby increases the complexity of cell structures that it is possible to culture on the scaffold assembly.

According to a third aspect of the invention there is provided a method of creating a scaffold, for use in tissue engineering to culture cells, comprising the steps of:
providing a base substrate; and
embroidering a plurality of interconnected stitches in the base substrate, each stitch being embroidered using a biocompatible thread.

This method shares the advantages of the scaffold.

Preferably the method of creating a scaffold includes providing a planar base substrate. The provision of a planar base substrate allows for the creation of a scaffold having embroidery stitches arranged in two-dimensions.

In another preferred embodiment of the invention the method of creating a scaffold includes providing a single, three-dimensional base substrate.

The method of creating a scaffold may include providing a plurality of planar base substrates overlying one another.

The provision of such three-dimensional base substrates allows for the creation of a three-dimensional scaffold.

Optionally the method of creating a scaffold includes the subsequent step of removing the base substrate from the plurality of interconnected stitches. Such a step avoids the need to remove the base substrate following, for example, implantation in a body.

According to a fourth aspect of the invention there is provided a, method of creating a scaffold assembly, for use in tissue engineering to culture cells, comprising the step of providing a plurality of layers overlying one another, each layer including a scaffold created according to any of the method steps set out herein above.

### BRIEF DESCRIPTION OF THE DRAWING

FIG. 1 shows three layers of a scaffold assembly according to one embodiment of the invention.

### DETAILED DESCRIPTION OF THE INVENTION

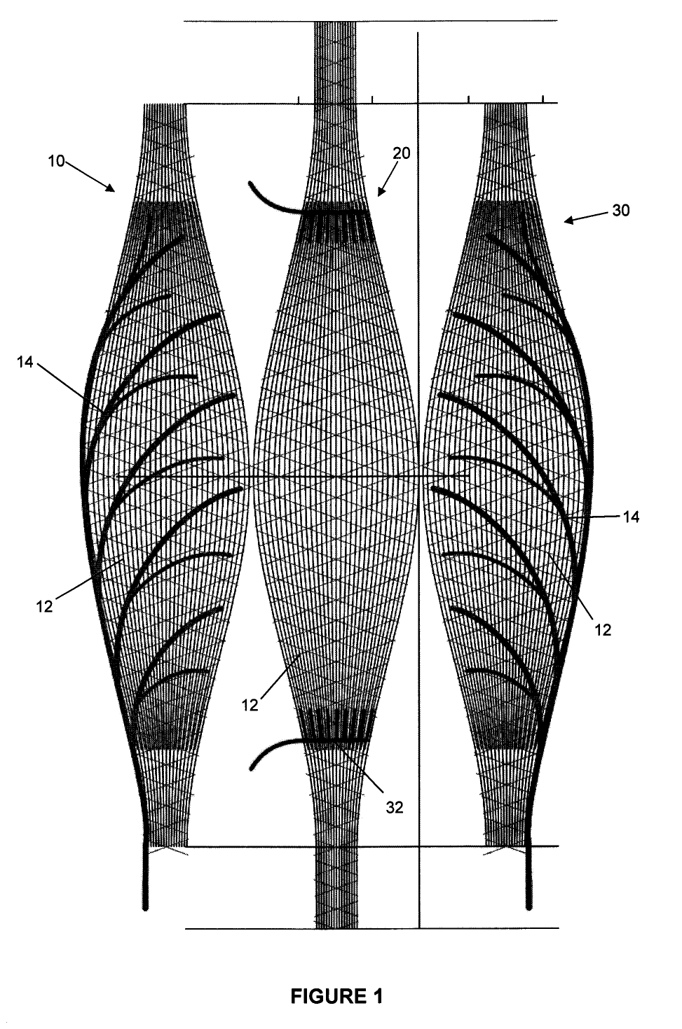

There now follows a brief description of preferred embodiments of the invention, by way of non-limiting examples, with reference to accompanying FIG. 1.

A scaffold, for use in tissue engineering to culture cells, according to a first embodiment of the invention is designated generally by the reference numeral 10.

The scaffold 10 includes a plurality of interconnected, first embroidery stitches 12, together with a plurality of interconnected, second embroidery stitches 14.

Each embroidery stitch 12, 14 is formed from a biocompatible thread 16.

In the embodiment shown, the first and second embroidery stitches 12, 14 are both lock stitches.

Other embodiments of the invention may include chain stitches, blind stitches, moss stitches, or tufts.

Those embodiments of the invention including a plurality of embroidered moss stitches have a significant third dimension which allows a scaffold including such stitches to culture a three-dimensional array of cells.

In those embodiments including a plurality of embroidered tufts, each tuft may be an I, J or W-shaped length of fibre, or a length of fibre in the form of a knot, the or each leg of the tuft forming a pile.

These tufts are formed by inserting a pile thread into a base substrate using a needle. The needle may be a tufting needle with an eye and a point at one end, or a fork needle. A fork needle includes a groove on one side to receive a thread, and may include a scarf on an opposite side. The scarf allows a hook to pass between the needle and a thread to catch the thread and form a loop. A knife may be used to cut each individual loop to form a tufted array.

The tufts may have legs of differing lengths. This is achieved by adjusting the spacing between the end of the loop and the base substrate.

A tufting head located above a computer-controlled or manually operated pantograph may be used to insert each tuft into a desired position within the base substrate. In other arrangements the tufting head may be moved relative to a stationary base substrate so as to allow the insertion of tufts into desired positions within the base substrate. Each of the foregoing arrangements allows for ready control of the density of tufts within different regions of a scaffold.

The biocompatible threads 16 may be resorbable. In other words, the biocompatible threads may be slowly degraded by biological and biochemical processes within a human or animal body, or slowly dissolve in a suitable solvent.

Examples of resorbable threads include threads including polylactic acid, polyglycoic acid, poly-L-lactide acid (PLLA), threads formed from protein fibres such as silks, and threads formed from glasses such as a combination of calcium phosphate and sodium phosphate.

Alternatively, the biocompatible threads 16 may be non-resorbable, such as polyester, nylon, polypropylene, or polyethylene threads, or metallic threads such as gold threads.

In each case the fibres from which the biocompatible threads 16 are made may be simple twisted fibres, braided fibres similar to a suture thread, or a gimp.

The biocompatible threads 16 may also include a bioactive component to promote the development of a specific type of cell. For example, the first embroidery stitches 12 are formed from a thread 16 including a bioactive component which promotes the growth of muscle cells.

In other embodiments of the invention (not shown) the scaffold includes a base substrate that supports the embroidery stitches. The base substrate may interconnect either discrete groups of interconnect embroidery stitches, or discrete embroidery stitches, such as tufts.

The base substrate may also be biocompatible so that it can remain in place during culture of any cells on the scaffold, or after implantation in a body of a scaffold complete with cultured cells grown thereon.

Examples of biocompatible substrates include biological substrates such as skin, tissue, or connective tissue.

The base substrate may be resorbable. Examples of resorbable base substrates include those made from collagen, collagen derivatives, or mineralized collagen. In addition, biopolymers such as chitosan, hyaluronic acids, and resorbable polyesters such as polylactone, poly-lactide, polyhydroxbutyrate, polycaprolactone, together with all of their co-polymers and derivatives, may also be used to form a resorbable base substrate.

Alternatively the scaffold may include a non-resorbable base substrate, such as a substrate formed from a metal, a ceramic, glass or carbon fibres, a plastic such as polyester, polytetrafluoroethylene (PTFE), polypropylene, polyethylene and nylon, or a silk fibre.

FIG. 1 shows two further layers of second and third scaffolds 20, 30. The third scaffold 30 is a mirror image of the first scaffold 10, while the second scaffold 20 includes a plurality of interconnected, first embroidery stitches 12 together with a plurality of interconnected, third embroidery stitches 32.

The patterns of embroidery stitches 12, 14, 32 on each scaffold 10, 20, 30 are coordinated with one another such that the scaffolds 10, 20, 30 may be stacked upon one another so as to define a scaffold assembly having a desired three-dimensional array of embroidery stitches 12, 14, 32.

The scaffold assembly includes scaffolds 10, 20, 30 having embroidery stitches 12, 14, 32 which abut and lie adjacent to one another so as to allow for the growth of all the differing cell types required to produce, for example, a muscle.

A preferred method of creating a scaffold includes the steps of providing a base substrate and embroidering a plurality of interconnected stitches 12, 14, 32 in the base substrate. Each stitch 12, 14, 32 is embroidered using a biocompatible thread 16.

Each biocompatible thread 16 may include a bioactive component, or fibres that include a bioactive component.

In other embodiments of the method one or more bioactive components may be applied to the scaffold by, for example, spraying using an inkjet printer head.

A planar base substrate may be provided so as to facilitate the creation of a scaffold including a two-dimensional array of embroidery stitches 12, 14, 32. Examples of planar base substrates include textile materials and simple films, such as a polyvinyl alcohol (PVA) film.

A single, three-dimension base substrate may be provided. Alternatively, a three-dimensional base substrate may be formed from a plurality of planar substrates stacked together. Such three-dimensional substrates permit the creation of a three-dimensional array of embroidery stitches 12, 14, 32 which can be exposed following removal of the base substrate. Such a three-dimensional array of embroidery stitches 12, 14, 32 closely resembles a complex cell structure and so facilitates the growth of such structures.

As indicated above, a method of the invention may include the step of removing the base substrate from the plurality of interconnected stitches 12, 14, 32.

An approximately configured arrangement of interconnected embroidery stitches is able to retain its integrity following removal of the base substrate.

The process of removing the base substrate varies according to the nature of the substrate. For example, a cotton, cellulose, or cellulosic base substrate could be removed by placing the scaffold in an acid, a PVA base substrate could be removed by washing in hot water, and an acetate base substrate could be removed by dissolving in acetone.

Other base substrates may be heat destructible.

A method of creating a scaffold assembly according to an embodiment of the invention includes the step of providing a plurality of layers overlying one another, each layer including a scaffold created as sat out above.

Each scaffold may be cultured with cells before stacking over another scaffold, or cells may be cultured on an already assembled scaffold assembly.

Cell culturing may take place wholly or partially within a culture system, or wholly or partially within a human or animal body.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A scaffold, for use in tissue engineering to culture cells, comprising a plurality of interconnected embroidery stitches, each stitch being formed from a biocompatible thread.

2. A scaffold according to claim 1 wherein the embroidery stitches include one or more from the group consisting of a lock stitch, a chain stitch, a blind stitch, a moss stitch, and a tuft.

3. A scaffold according to claim 2 including a plurality of tufts having legs of differing lengths.

4. A scaffold according to claim 1 wherein the biocompatible thread is resorbable.

5. A scaffold according to claim 1 wherein the biocompatible thread is non-resorbable.

6. A scaffold according to claim 1 wherein the biocompatible thread includes a bioactive component to promote the development of a specific type of cell.

7. A scaffold according to claim 1 further including a base substrate supporting the embroidery stitches.

8. A scaffold according to claim 7 wherein the base substrate interconnects the embroidery stitches.

9. A scaffold according to claim 8 including a biocompatible base substrate.

10. A scaffold according to claim 7 wherein the base substrate is resorbable.

11. A scaffold assembly, for use in tissue engineering to culture cells, comprising a plurality of layers overlying one another, each layer including a scaffold according to claim 1.

12. A method of creating a scaffold, for use in tissue engineering to culture cells, comprising the steps of:
providing a base substrate; and
embroidering a plurality of interconnected stitches in the base substrate, each stitch being embroidered using a biocompatible bead.

13. A method of creating a scaffold according to claim 12 including providing a planar base substrate.

14. A method of creating a scaffold according to claim 12 including providing a single, three-dimensional base substrate.

15. A method of creating a scaffold according to claim 12 including providing a plurality of planar base substrates overlying one another.

16. A method of creating a scaffold according to claim 12 including applying one or more bioactive components to the scaffold.

17. A method of creating a scaffold according to claim 12 including the step of removing the base substrate from the plurality of interconnected stitches.

18. A method of creating a scaffold assembly, for use in tissue engineering to culture cells, comprising the step of providing a plurality of layers overlying one another, each layer including a scaffold created according to the method of claim 12.
